(19) United States
(12) Patent Application Publication
(10) Pub. No.: US 2005/0008668 A1
(43) Pub. Date: Jan. 13, 2005

(54) Carbamates for the modification of rheological properties

(76) Inventors: Angelo Provinzano, Bottrop (IT); Jurgen Meyer, Essen (DE); Wolfgang Goertz, Essen (DE)

Correspondence Address: Leopold Presser;Scully, Scott, Murphy & Presser, 400 Garden City Plaza, Garden City, NY, 11530, US

(21) Appl. No.: 10/883,411

(22) Filed: Jul. 1, 2004

(30) Foreign Application Priority Data
Jul. 9, 2003 (DE) 103 31 015.0

(51) Int. Cl. A61K 7/18; C08G 18/70; A61K 7/06; A61K 7/11

(52) U.S. Cl. 424/401; 424/70.17; 528/67

(57) ABSTRACT

Carbamates obtainable by reacting a) at least one isocyanate of the general formula (II) and/or (IIa)
(OCN—R¹—NCO) (II)
(R⁴—NCO) (IIa) b) at least one hydroxyl compound of the general formula (III) and/or (IIIa) and/or (IIIb)
(R²—(R³O)ₙ—OH) (III)
HO—CH₂—(CH—OH)ₘ—CH₂—OH (IIIa)
R—O—[Z]_{b} (IIIb) are described as well as their use for the modification of Theological properties of homogeneous-phase systems or hetergeneous-phase systems which contain at least one hydrophobic component.

### FIELD OF THE INVENTION

The present invention relates to carbamates and their use for the modification of Theological properties of homogeneous-phase or heterogeneous-phase systems, which contain at least one hydrophobic component.

### BACKGROUND OF THE INVENTION

In the preparation of cosmetic, pharmaceutical and technical products, hydrophobic substances are often used. Usually, these substances are liquid at room temperature. Such liquids are, for example, aliphatic or cycloaliphatic hydrocarbons or hydrocarbon mixtures, oils, fats or other organic solvents, which are not soluble or only very slightly soluble in water.

Cosmetic, pharmaceutical and technical formulations should usually have specific rheological property profiles and can therefore often only be brought into the desired application form with the aid of additives, e.g., “thickeners”. Examples of known low molecular weight thickening agents for oil phases are, for example, alkali metal and aluminum salts of fatty acids, long-chain fatty alcohols, long-chain amines, waxes, and also amino acid or steroid derivatives.

Often, the use of thickeners of this type is, however, associated with disadvantages for the systems to be thickened. Thus, the thickening power of the additive employed can be too low or a desired rheological profile cannot be achieved. Furthermore, the use of the known substances in cosmetic or pharmaceutical applications is often not desirable for health or sensory reasons. At the same time, it is to be observed that, particularly in formulations for the application areas mentioned above, the demand is becoming stronger and stronger to keep the proportion of inactive substances, such as thickeners, as low as possible.

Often, polymers are employed for thickening hydrophobic, water-insoluble liquids, which in general allow an adjustment of the viscosity by variation of the molecular weight, provided these polymers are adequately soluble in the solvent to be thickened.

As thickeners, polymers, however, can have the disadvantage that with increasing molecular weight incorporation is made difficult. Thus, often only a swelling of the polymer instead of the desired homogeneous solution in the system to be thickened can be observed. Therefore, for the preparation of such formulations solvents must be employed as auxiliaries that subsequently can no longer be removed completely from the system. This restricts the utility of these polymer thickeners for specific applications (cosmetic, pharmaceutical, or technical).

Furthermore, it is to be observed in this connection that polymers as a rule always possess a wide molecular weight distribution, which restricts the effectiveness as thickeners and moreover can be associated with disadvantages in their use in cosmetic, pharmaceutical or technical formulations or can even make this use impossible.

The use of ester-terminated polyamides for the formulation of transparent gels from liquid hydrocarbons having a low polarity is described, for example, in WO-A-98/17243 and WO-A-98/17705. It is disadvantageous in the case of these thickeners that the gels thus produced yellow with time. Moreover, various synthetic oils, such as, for example, silicone oils, cannot be thickened by these additives.

In WO-A-97/36572, a base system for the preparation of cosmetic formulations is described which consists of at least one liquid silicone and at least one gel-forming additive. The additives employed in WO '572 are polymers which contain siloxane groups. Moreover, polar structures such as ester groups, urethane groups, urea groups, thiourea groups or amide groups, which are suitable for the formation of hydrogen bonds and can thereby thicken the base formulation, are incorporated into these additives. However, the polymer thickeners described are only very moderately effective. Thus, cosmetic compositions in stick form can only be formulated with the use of excessively large amounts of thickener (about 60% based on the total formulation). Moreover, the polymers employed as thickeners are not suitable for silicone-free oils. The thickening of mixtures of silicone oils and other oils is not described in this specification.

WO-A-97/36573 also describes a base system for the preparation of cosmetic compositions, which contains at least one liquid silicone and a gel-forming agent based on a combination of a wax and of a polyamide. In this composition, a wax and/or a polymer is modified by siloxane groups. The thickening mixture is therefore aimed specifically at use in formulations having a high silicone oil content. Here too, the thickening of mixtures of silicone oils and other oils or the thickening of non-silicone-containing oil systems is not described.

In WO-A 01/85821, a polyurethane is described which is synthesized from at least one diisocyanate, at least one polyether having two active hydrogen atoms per molecule and at least one compound of the general formula R¹—Z¹, in which R¹ is C₈- to C₃₀-alkyl or -alkenyl or analogous aryl-containing compounds and Z¹ is a hydroxyl, thiol or alkylamine group. The polyurethanes described in WO '821 preferably have 2 to 50, in particular 3 to 45, urethane and/or urea groups per molecule. The urethanes thus described are able, as components, to thicken homogeneous and heterogeneous formulations which contain at least one water-insoluble compound liquid at 20° C. Furthermore, the urethanes described are generally able to thicken oils, fats, saturated hydrocarbons, ester oils, and silicone oils and mixtures of these oils. The application of these polyurethanes in cosmetic, pharmaceutical and industrial applications is furthermore described.

A disadvantage in the use of the polyurethanes described in WO '821 is that on account of the synthesis of the products from isocyanates with polyethers and, for example, alcohols, a multiplicity of reaction products is undeniably obtained. This has the result that, in addition to oil-thickening components having a high activity, large amounts having a lower activity are obtained. Furthermore, and particularly for cosmetic and pharmaceutical applications, additives are desired which have a well-defined, known structure and composition. In this sense, the use of polyurethanes such as are described in WO-A-01/85821 is clearly rendered difficult in these applications.

There is thus a need to develop effective oil thickeners having a defined chemical structure and without a multiplicity of possible by-products. These thickeners should effectively thicken oils and other liquid hydrophobic compounds and should be capable of being readily incorporated into the systems to be thickened. At the same time, the thickened systems should maintain their high consistency up to a temperature of 45° C. Furthermore, they should also be able to stabilize W/O emulsions by thickening the oil phase.

### SUMMARY OF THE INVENTION

An object of the present invention is thus to make available effective thickeners for liquid hydrophobic compounds, which are able to fulfill the criteria mentioned above.

Surprisingly, it has now been found that these objects can be fulfilled in an outstanding manner by low molecular weight carbamate thickeners which can be synthesized in a very flexible and at the same time very defined manner from two alkyl polyethers and a diisocyanate or from polyhydroxy compounds such as carbohydrates and isocyanates.

One aspect of the present invention is therefore carbamates that are obtainable by reacting

at least one isocyanate of the general formula (II) and/or (IIa)
(OCN—R¹—NCO) (II)
(R⁴—NCO) (IIa)
in which
R¹, R⁴ independently of one another is an aliphatic, cycloaliphatic and/or aromatic hydrocarbon radical, with
at least one hydroxyl compound of the general formula (III) and/or (IIIa) and/or (IIIb)
(R²—(R³O)ₙ—OH) (III)
HO—CH₂—(CH—OH)ₘ—CH₂—OH (IIIa)
R—O—[Z]_{b} (IIIb)
in which
R² can be a branched or straight-chain C₈-C₅₀-alkyl or branched or straight-chain C₈-C₅₀-alkenyl radical or a C₆-C₂₀-aryl branched or straight-chain alkyl or alkenyl radical,
R³ can be CₐH₂ₐ where a=2 to 12, preferably a=2,

R can be alkyl or alkenyl radicals having 6 to 22 C atoms or H,

Z can be a sugar radical having 5 or 6 carbon atoms,

b can be 1 to 10,

n can be 0 to 200, preferably 5 to 50, and

m can be 1 to 4.

In one embodiment of the present invention, compounds of the general formula (I)
R²—(R³O)ₙ—O—C(O)—N(H)—R¹—N(H)—C(O)—O—(OR³)ₙ—R² (I)

are prepared. Specifically, the compounds of general formula (I) are prepared by reacting

at least one diisocyanate of the general formula (II)
(OCN—R¹—NCO) (II)
wherein R¹ is an aliphatic, cycloaliphatic and/or aromatic hydrocarbon radical, with alkyl polyethers of the general formula (III)
(R²—(R³O)ₙ—OH) (III)
in which
R² can be a branched or straight-chain C₈-C₅₀-alkyl or branched or straight-chain C₈-C₅₀-alkenyl radical or a C₆-C₂₀-aryl branched or straight-chain alkyl or alkenyl radical,
R³ can be CₐH₂ₐ where a=2 to 12, preferably a=2.

The present invention also relates to the use of at least one of the inventive carbamates as a component of a homogeneous-phase or heterogeneous phase composition that contains at least one water-soluble, liquid compound for the modification of rheological properties. The present invention also provides a composition that includes at least of the inventive carbamates and at least one water-insoluble, liquid composition.

Additionally, the present invention relates to the use of at least one of the inventive carbamates as or in a cosmetic or pharmaceutical preparation, as or in a coating and treatment agent for non-absorbent surfaces such as, for example, metals, plastics, or textile fibers and glass, and also for absorbent surfaces such as, for example, wood, paper, cotton or leather.

A yet further aspect of the present invention relates to the use of a composition of the present invention in the production of candles, fuels, motor fuels, industrial fats, antirust agents and inkjet printer cartridges.

The present invention also provides a cosmetic or pharmaceutical composition that include at least one of the inventive carbamates, at least one water-insoluble, liquid compound, at least one cosmetically or pharmaceutically active substance, and optionally, at least one additive.

### DETAILED DESCRIPTION OF THE INVENTION

The present invention, which relates to carbamates and their use for modifying rheological properties of homogeneous-phase or heterogeneous-phase systems that include at least one hydrophobic component, will now be described in greater detail.

The preparation of the carbamates according to the present invention is preferably carried out by
(A) reaction of one mol of diisocyanate (e.g., hexamethylene diisocyanate) with 2 mol of an alkyl polyether (e.g., C₃₀H₆₁(EO)₁₃H).

First, the appropriate amount of the alkyl polyether is weighed in a solvent, such as, toluene, and slowly heated under reflux for 30 minutes in a nitrogen atmosphere for drying. The solvent is subsequently stripped off, the mixture is cooled to 80° C., a catalyst is added (dibutyltin dilaurate) and finally the appropriate amount of diisocyanate is briskly added dropwise. The course of the reaction can be observed and checked by determination of the NCO number.

Furthermore, the carbamates according to the present invention can be prepared by
(B) reaction of polyhydroxy compounds, for example of a sugar alcohol and/or of a sugar (e.g., one mol of glucose) with the appropriate amount of an isocyanate (e.g., five mol of stearyl isocyanate).

In the example mentioned above, glucose and toluene are weighed in at the same time and heated to boiling under nitrogen. For the drying of the glucose, the contents of the flask are heated under reflux (at 110° C.) with an attached water separator for 30 minutes. Subsequently, the mixture is cooled to 100° C. and the catalyst (dibutyltin dilaurate) is added and finally the isocyanate is briskly added dropwise. Again, the course of the reaction can be monitored by checking the NCO number.

Subsequently, the solvent is stripped off and the corresponding carbamate is isolated.

A further subject of the present invention is the use of at least one carbamate, as defined above, as a component of a homogeneous- or heterogeneous-phase composition, which contains at least one water-insoluble, liquid compound, the composition being mixed with at least one carbamate, as also described above, and optionally heated.

The “modification of Theological properties” is widely understood here in the context of the present invention. The carbamates according to the present invention are generally suitable for the thickening of the consistency of hydrophobic compounds in a wide range. According to the basic consistency of the hydrophobic liquid compounds to be thickened, and depending on the amount of carbamate used, as a rule the flow properties can be modified from highly liquid to gelatinous (solid-like). “Modification of rheological properties” is therefore understood, inter alia, as meaning an increase in the viscosity of liquids, the improvement in the thixotropic properties of gels, and the solidification of gels and waxes.

“Homogeneous-phase compositions” in this case consist, independently of the number of their constituents, of only one phase (are thus homogeneous). “Heterogeneous-phase compositions” are disperse systems of two or more phases, which can be constructed of as many components as desired. These preferably include emulsions, such as, for example, O/W or W/O formulations, which contain at least one oil or fat component and water as the immiscible phases. The carbamates according to the present invention are as a rule employed in the oil phase.

The compositions according to the present invention contain at least one essentially water-insoluble (e.g., hydrophobic) liquid (oil or fat component). “Water-insoluble” is understood as meaning a water solubility of in general at most 1 g/l at 20° C.

The compositions according to the present invention preferably have at least one such water-insoluble oil or fat component, which is preferably selected from:
oils, preferably mineral oils, fully synthetic oils, oils of vegetable and animal origin and ethereal oils,
fats,
saturated acyclic and cyclic hydrocarbons,
esters of monocarboxylic acids with mono-, di- or trihydric alcohols,
silicone oils and mixtures thereof.

The compositions according to the present invention have, for example, an oil or fat component which is selected from:

hydrocarbons of low polarity, such as, for example, mineral oils; linear saturated hydrocarbons, such as, for example, tetradecane, hexadecane, octadecane, and the like; cyclic hydrocarbons, such as, for example, decahydronaphthalene; branched hydrocarbons; esters, preferably esters of fatty acids, such as, for example, esters of C₁- to C₂₂-monoalcohols with C₁- to C₂₂-monocarboxylic acids, such as, for example, isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, and tetratriacontanyl stearate; salicylates, such as C₁- to C₁₀-salicylates, e.g., octyl salicylate; benzoate esters, such as C₁₀- to C₁₅-alkyl benzoates, benzyl benzoate; other cosmetic esters, such as fatty acid triglycerides, propylene glycol monolaurate, polyethylene glycol monolaurate, castor oil, C₁₀- to C₁₅-alkyl lactates, etc.

The compositions according to the present invention contain these oil or fat components in general in an amount from 1 to 99.9% by weight, preferably 10 to 90% by weight, in particular 20 to 80% by weight, based on the total weight of the composition.

The compositions according to the present invention are thus preferably suitable for the formulation of oil- or fat-containing cosmetic, pharmaceutical and technical products. According to the basic consistency of the oil or fat components, the properties can be varied from highly liquid up to a solid consistency of the composition depending on the amount of carbamate used.

A further subject of the instant invention is the use of compositions, such as defined hereinabove, as or in cosmetic and pharmaceutical preparations, as or in coating and treatment agents for nonabsorbent surfaces, preferably metals, plastics, textile synthetic fibers and glass, and for absorbent surfaces, preferably wood, paper, cotton and leather.

A further subject of the present invention is the use of compositions, such as defined hereinabove, for the production of candles, fuels and motor fuels, industrial fats, antirust agents and inkjet printer cartridges.

A further subject of the instant invention is the use of carbamates according to the present invention as components of pharmaceutical, cosmetic and technical preparations, preferably in cosmetic preparations for the treatment of the skin, and also for the modification of rheological properties of compositions based on compounds of low polarity.

A further subject of the present invention is a cosmetic or pharmaceutical composition comprising:
at least one carbamate, as defined above,
at least one water-insoluble, liquid compound,
at least one cosmetically or pharmaceutically active substance, and
optionally at least one additive.

According to a preferred embodiment of the present invention, the cosmetic compositions according to the invention are present in the form of an oil-containing or fat-containing cosmetic preparation. These include, for example, creams, lotions, baby oil, bath oil, mascara, eye make-up, face make-up, cosmetic oils, make-up remover, skin moisturizing agents, sunscreens, lip care compositions, water-free handwashes, medicinal ointments, and the like.

The oil- or fat-containing cosmetic compositions according to the present invention contain the corresponding oil or fat components in an amount of from approximately 1 to 99.9% by weight, preferably 10 to 90% by weight, in particular 20 to 80% by weight, based on the total weight of the composition.

The oil- or fat-containing cosmetic compositions according to the instant invention contain the carbamates described above in an amount from 0.1 to 50% by weight, preferably 0.2 to 30% by weight, in particular 0.5 to 10% by weight, based on the total weight of the composition.

Furthermore, the oil- or fat-containing cosmetic compositions can contain auxiliaries and/or additives, such as emulsifiers, superfatting agents, stabilizers, waxes, consistency-imparting agents, thickening agents, silicone compounds, biogenic active ingredients, film-forming agents, preservatives, hydrotropes, solubilizers, UV absorbers, colorants and fragrances, and the like.

The oil- or fat-containing cosmetic products according to the present invention based on the carbamates described above can in general be adjusted in a wide range with respect to their rheological properties. According to the basic consistency of the cosmetic composition, the properties can in general be varied from highly liquid up to a gelatinous consistency depending on the amount of the oligomer used. Advantageously, skin cosmetic products can thus be formulated which contain a high proportion of highly liquid oil- or fat-containing components.

Advantageously, the carbamates described and the compositions according to the present invention are suitable for the formulation of gels. The term “gel” is understood, in general, as meaning shape-stable substances, i.e., these substances are self-supporting to the extent that they are able to maintain their shape without a shape-stabilizing envelope (in contrast to liquids which would lose this shape as a result of flowing). In general, the oil or fat components mentioned herein are suitable for the formulation of such gels.

The inventive carbamates can, as a rule, be formulated with customary additives to give the cosmetic compositions according to the invention, such as, for example, lip care compositions, deodorants, antiperspirants, make-ups, etc. Advantageously, the carbamates described and the compositions according to the invention based on them can also be employed for the preparation of non-cosmetic products based on gels. These include, for example, automobile waxes and polishes, candles, furniture polishes, metal cleaners, household cleaners, etc.

Advantageously, the carbamates according to the instant invention and the compositions according to the present invention based on them are also suitable for the preparation of customary O/W and W/O emulsions, such as, for example, creams, the carbamates, as a rule, being employed in the oil phase.

The carbamates according to the present invention are particularly suitable as thickening agents for liquids of low polarity, preferably oils.

The carbamates and the compositions according to the present invention are preferably suitable for use in personal-care products, such as, for example, cosmetic compositions. They are furthermore advantageously suitable for the formulation of hair cosmetic products, such as hairsprays, foam setting lotions, hair mousse, hair gel and shampoos. They are furthermore preferably suitable for use in decorative cosmetics, in particular in mascara and eyeshadow. The carbamates described and their compositions according to the present invention can further be employed in household products, such as automobile waxes and polishes, candles, furniture polishes, metal cleaners, metal polishes, household cleaners, stain removers and carrier materials for insecticides. They are furthermore suitable for use in technical and industrial products, such as, for example, in motor fuels, fats, soldering pastes, antirust agents and inkjet printer cartridges.

The invention will be illustrated in greater detail by the following, non-restricting examples.

### EXAMPLES

Carbamate Preparation:

Carbamate 1:

A four-necked flask was flushed with nitrogen before initial weighing. 1.320 g of C₃₀H₆₁(C₂H₅₀)₅H (Unithox® 420 (Petrolite)) in 200 g of toluene were then weighed in and heated to boiling under a blanket of nitrogen. For the drying of Unithox® 420, the contents of the flask were heated under reflux for 30 minutes. The toluene was subsequently distilled off at 130° C. and 20 hPa. The reaction batch was subsequently cooled to 80° C. 0.75 g of dibutyltin dilaurate was added and the brisk dropwise addition of 171 g of hexa-methylene diisocyanate (Desmodur® H (BASF)) was initiated. The course of the reaction was checked by determination of the NCO number. The reaction was over after about three hours.

Carbamate 2:

18.0 g of dextrose and 200 g of toluene were weighed into a four-necked flask, which was flushed with nitrogen before initial weighing, and heated to boiling under nitrogen. For the drying of the glucose, the contents of the flask were heated under reflux (110° C.) with a water separator for 30 min. The mixture was subsequently cooled to 100° C. 0.25 g of dibutyltin dilaurate were then added and the brisk dropwise addition of 147.8 g of stearyl isocyanate was initiated (at 100° C. under a nitrogen atmosphere). The course of the reaction was checked by determination of the NCO number. The reaction was over after about one hour.

Application Technology:

The carbamates according to the present invention had a strongly thickening action in paraffin oil, mineral oil, and also in nonpolar triglycerides (olive, avocado, soybean or jojoba oil). Emollient esters such as octyl palmitate or octyl stearate were also thickened. Furthermore, aqueous mixtures of these oils with portions of water were also well thickened.

This thickening occurred even at carbamate contents of about 5% by weight. The thickened oil phases obtained were relatively clear and stable up to 45° C.

The following viscosities were obtained, for example, by addition of 5% by weight of carbamate 1 to various oils (measured at 2 s⁻¹ (cone/plate) (25° C.)):

(viscosity of the pure oil in brackets as η_{LM})

260 Pas (in paraffin oil (η_{LM} about 30 mPas)), 50 Pas (mineral oil (η_{LM} about 28 mPas)), 30 Pas (caprylic/capric triglyceride (η_{LM} about 25 mPas).

By addition of 5% by weight of carbamate 2, for example, paraffin oil (112 Pas) or caprylic-capric triglyceride (100 Pas) was strongly thickened.

Moreover, the carbamates according to the present invention were employed, for example, in the formulation of a specific two-phase oil-containing foam bath containing a proportion of 30% by weight of triglycerides (carbamate content 1 to 10% by weight) (thickening of the oil phase).

While the present invention has been particularly shown and described with respect to preferred embodiments, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

1. A carbamate obtainable by reacting
a) at least one isocyanate of the general formula (II), (IIa), or a combination thereof
(OCN—R¹—NCO) (II)
(R⁴—NCO) (IIa)
in which
R¹, R⁴ independently of one another is an aliphatic hydrocarbon radical, a cycloaliphatic hydrocarbon radical, an aromatic hydrocarbon radical or a mixture thereof, with
b) at least one hydroxyl compound of the general formula (III), (IIIa), (IIIb), or a combination thereof
(R²—(R³O)ₙ—OH) (III)
HO—CH₂—(CH—OH)ₘ—CH₂—OH (IIIa)
R—O—[Z]_{b} (IIIb)
in which
R² is a branched or straight-chain C₈-C₅₀-alkyl, a branched or straight-chain C₈-C₅₀-alkenyl radical, or a C₆-C₂₀-aryl branched or straight-chain alkyl or alkenyl radical,
R³ is CₐH₂ₐ where a=2 to 12,
R is an alkyl or alkenyl radical having 6 to 22 C atoms or H,
Z is a sugar radical having 5 or 6 carbon atoms,
b is 1 to 10,
n is 0 to 200, and
m is 1 to 4.

2. A carbamate as claimed in claim 1, wherein a is 2.

3. A carbamate as claimed in claim 1, wherein n is 5 to 50.

4. A carbamate as claimed in claim 1, comprising compounds of the general formula (I)
R²—(R³O)ₙ—O—C(O)−N(H)—R¹—N(H)—C(O)—O—(OR³)ₙ—R² (I)
which are obtainable by reacting
a) at least one diisocyanate of the general formula (II)
(OCN—R¹—NCO) (II)
R¹ is an aliphatic hydrocarbon radical, a cycloaliphatic hydrocarbon radical, an aromatic hydrocarbon radical or a mixture thereof, with
b) alkyl polyethers of the general formula (III)
(R²—(R³O)ₙ—OH) (III)
in which
R² is a branched or straight-chain C₈-C₅₀-alkyl, a branched or straight-chain C₈-C₅₀-alkenyl radical, or a C₆-C₂₀-aryl branched or straight-chain alkyl or alkenyl radical,
R³ is CₐH₂ₐ where a=2 to 12.

5. A carbamate as claimed in claim 4, wherein a is 2.

6. A carbamate as claimed in claim 1, obtainable by reacting at least
a) one isocyanate of the general formula (IIa) and optionally (II)
(OCN—R¹—NCO) (II)
R⁴—NCO) (II a)
in which
R¹, R⁴ independently of one another is an aliphatic hydrocarbon radical, a cycloaliphatic hydrocarbon radical, an aromatic hydrocarbon radical or a mixture thereof, with
b) at least one hydroxyl compound of the general formula (IIIa), (IIIb), or a mixture thereof
HO—CH₂—(CH—OH)ₘ—CH₂—OH (IIIa)
R—O—[Z]_{b} (II)
in which
R is an alkyl or alkenyl radical having 6 to 22 C atoms or H,
Z is a sugar radical having 5 or 6 carbon atoms,
b is 1 to 10,
n is 0 to 200, and
m is 1 to 4.

7. A carbamate as claimed in claim 1, wherein the isocyanate according to general formula (II) additionally used is at least one compound selected from hexamethylene diisocyanate, tetra-methylene diisocyanate, isophorone diisocyanate, or dicyclohexylmethane-4,4′-diisocyanate.

8. A carbamate as claimed in claim 1, wherein the isocyanate according to general formula (IIa) additionally used is at least one compound in which the radical R⁴ is an optionally branched alkyl radical having 6 to 22 C atoms.

9. A carbamate as claimed in claim 4, wherein the hydroxyl compound according to general formula (III) additionally used is at least one compound in which R² is a branched or straight-chain C₈-C₂₂-alkyl or alkenyl radical and R³ is the radical —(C₂H₄O)ₙ—, where n=5 to 15.

10. A carbamate as claimed in claim 6, wherein the hydroxyl compound according to general formula (IIIa) additionally used is at least one compound in which m=2 to 4.

11. A carbamate as claimed in claim 6, wherein the hydroxyl compound according to general formula (IIIb) additionally used is at least one compound in which Z is at least one sorbitol, glucose or fructose radical.

12. A homogeneous-phase or heterogeneous-phase composition comprising at least one water-insoluble, liquid compound, and a carbamate as claimed in claim 1 for the modification of the rheological properties of the composition.

13. A composition consisting of at least one carbamate as claimed in claim 1 and at least one water-insoluble, liquid compound selected from the group consisting of oils, fats, saturated acyclic and cyclic hydrocarbons, esters of monocarboxylic acids with mono-, di- or trihydric alcohols, silicone oils and their mixtures with one another and among one another.

14. A cosmetic or pharmaceutical composition comprising at least one carbamate as claimed in claim 1, at least one water-insoluble, liquid compound, at least one cosmetically or pharmaceutically active substance and optionally at least one additive.

15. A method of preparing a carbamate comprising reacting
a) at least one isocyanate of the general formula (II), (IIa), or a combination thereof
(OCN—R¹—NCO) (II)
(R⁴—NCO) (IIa)
in which
R¹, R⁴ independently of one another is an aliphatic hydrocarbon radical, a cycloaliphatic hydrocarbon radical, an aromatic hydrocarbon radical or a mixture thereof, with
b) at least one hydroxyl compound of the general formula (III), (IIIa), (IIIb), or a combination thereof
(R²—(R³O)ₙ—OH) (III)
HO—CH₂—(CH—OH)ₘ—CH₂—OH (IIIa)
R—O—[Z]_{b} (IIIb)
in which
R² is a branched or straight-chain C₈-C₅₀-alkyl, a branched or straight-chain C₈-C₅₀-alkenyl radical, or a C₆-C₂₀-aryl branched or straight-chain alkyl or alkenyl radical,
R³ is CₐH₂ₐ where a=2 to 12,
R is an alkyl or alkenyl radical having 6 to 22 C atoms or H,
Z is a sugar radical having 5 or 6 carbon atoms,
b is 1 to 10,
n is 0 to 200, and
m is be 1 to 4.

16. The method of claim 15 wherein a) is at least one diisocyanate of the general formula (II)
(OCN—R¹—NCO) (II)
R¹ is an aliphatic hydrocarbon radical, a cycloaliphatic hydrocarbon radical, an aromatic hydrocarbon radical or a mixture thereof, and
b) is alkyl polyethers of the general formula (III)
(R²—(R³O)ₙ—OH) (III)
in which
R² is a branched or straight-chain C₈-C₅₀-alkyl, a branched or straight-chain C₈-C₅₀-alkenyl radical, or a C₆-C₂₀-aryl branched or straight-chain alkyl or alkenyl radical,
R³ is CₐH₂ₐ where a=2 to 12.

17. The method of claim 15 wherein a) is at least one isocyanate of the general formula (IIa) and optionally (II)
(OCN—R¹—NCO) (II)
(R⁴—NCO) (II a)
in which
R¹, R⁴ independently of one another is an aliphatic hydrocarbon radical, a cycloaliphatic hydrocarbon radical, an aromatic hydrocarbon radical or a mixture thereof, and
b) is at least one hydroxyl compound of the general formula (IIIa), (IIIb), or a mixture thereof
HO—CH₂—(CH—OH)ₘ—CH₂—OH (IIIa)
R—O—[Z]_{b} (IIIb)
in which
R is an alkyl or alkenyl radical having 6 to 22 C atoms or H,
Z is a sugar radical having 5 or 6 carbon atoms,
b is 1 to 10,
n is 0 to 200, and
m is 1 to 4.

18. The method of claim 15, wherein the isocyanate according to general formula (II) additionally used is at least one compound selected from hexamethylene diisocyanate, tetra-methylene diisocyanate, isophorone diisocyanate, or dicyclohexylmethane-4,4′-diisocyanate.

19. The method of claim 15, wherein the isocyanate according to general formula (IIa) additionally used is at least one compound in which the radical R⁴ is an optionally branched alkyl radical having 6 to 22 C atoms.

20. The method of claim 16, wherein the hydroxyl compound according to general formula (III) additionally used is at least one compound in which R² is a branched or straight-chain C₈-C₂₂-alkyl or alkenyl radical and R³ is the radical —(C₂H₄O)ₙ—, where n=5 to 15.

21. The method of claim 17, wherein the hydroxyl compound according to general formula (IIIa) additionally used is at least one compound in which m=2 to 4.

22. The method of claim 17, wherein the hydroxyl compound according to general formula (IIIb) additionally used is at least one compound in which Z is at least one sorbitol, glucose or fructose radical.
